(19) United States
(12) Patent Application Publication
(10) Pub. No.: US 2010/0040549 A1
(43) Pub. Date: Feb. 18, 2010

(54) Composition for Targeted Drug Delivery and Controlled Release

(75) Inventors: Nancy J. Halas, Houston, TX (US); Dongmao Zhang, Houston, TX (US); Aoune Barhoumi, Houston, TX (US)

Correspondence Address: CONLEY ROSE, P.C., 5601 GRANITE PARKWAY, SUITE 750, PLANO, TX, 75024, US

(73) Assignee: WILLIAM MARSH RICE UNIVERSITY, Houston, TX (US)

(21) Appl. No.: 12/520,978

(22) PCT Filed: Jan. 4, 2008
(86) PCT No.: PCT/US2008/050301

§ 371 Date: Jun. 23, 2009

Related U.S. Application Data
(60) Provisional application No. 60/883,599, filed on Jan. 5, 2007.

(51) Int. Cl. A61K 49/00; A61K 9/16; A61K 31/7088; A61K 38/28

(52) U.S. Cl. 424/9.1; 424/490; 514/44 R; 514/3; 514/44 A

(57) ABSTRACT

Disclosed herein are novel targeted drug delivery and controlled release methods and compositions where optically absorbing nanoparticles, such as nanoshells, are functionalized on their surfaces with thermolabile molecules that bind the drug molecules to be delivered. The linkage between the thermolabile moiety on the nanoparticles and the drug is deliberately designed or selected to be temperature sensitive, so that upon illumination of the nanoparticle at a wavelength of light, the drug molecules on the nanoparticles will be released. Targeting molecules, such as antibodies, aptamers or other molecules like folic acid, can be concurrently bound to the nanoparticle surface to deliver the nanoparticle to specifically targeted cells or tissues prior to the photothermally induced drug release. In this way the nanoparticles can be advantageously concentrated on the target prior to illumination, which makes the disclosed compositions both a targeted delivery and a controllable drug release vehicle.

### CROSS-REFERENCE TO RELATED APPLICATIONS

This application is a filing under 35 U.S.C. 371 of International Application No. PCT/US2008/050301 filed Jan. 4, 2008, entitled “Composition for Targeted Drug Delivery and Controlled Release,” claiming priority of U.S. Provisional Application No. 60/883,599 filed Jan. 5, 2007, which applications are incorporated by reference herein in their entirety.

### STATEMENT REGARDING FEDERALLY SPONSORED RESEARCH OR DEVELOPMENT

This work was supported by the National Science Foundation (NSF) Grant EEC-0304097, AFOSR F49620-03-C-0068, Robert A. Welch Foundation C-1220, the Multidisciplinary University Research Initiative (MURI) of the Department of Defense W911NF-04-01-0203, and the Department of Defense Breast Cancer Research Program DAMD17-03-1-0384, and the Texas Institute for Bio-Nano Materials and Structures for Aerospace Vehicles funded by NASA.

### BACKGROUND OF THE INVENTION

This invention relates to generally to the field of drug delivery. More specifically, the invention relates to a composition for targeted and controlled release of drugs.

Drug delivery for targeted and controlled release is very important in medical applications for its potential in ultimately reducing drug cytotoxicity and improving drug bioavailability. Currently, the most common approach for targeted release is through micelles, di- or tri block polymeric colloidal core-shell structures, as delivery carriers, where drug molecules are encapsulated in the core and the shell of the colloid is functionalized for specific targets. Although this technique promises targeted delivery, there is no clear mechanism for direct, externally controlled drug release. In fact, because it must maintain stability under physiological conditions, the shell layer of the drug-containing micelle may actually hinder release of the drug from the core.

Much attention has been focused on the so-called genetic metabolic diseases in which a defective gene causes an enzyme to be either absent or ineffective in catalyzing a particular metabolic reaction effectively. A potential approach to the treatment of genetic disorders is gene therapy. This is a technique whereby the absent or faulty gene is replaced by a working gene, so that the body can make the correct enzyme or protein and consequently eliminate the root cause of the disease or alternatively, prevent a cell from overproducing an enzyme or protein.

In conventional gene therapy, a single strand (ss) DNA or RNA oligomer is administered into human body with a hope that it binds to a specific DNA or RNA target to generate a therapeutic effect through modifying the expression of a specific gene. However, because of the instability of ss DNA or short interfering RNA (siRNA) in the physiological environment, the half-life of such molecules in vivo is less than 5 minutes, which greatly limits the therapeutic effect of gene therapy.

Consequently, there is a need for compositions and methods for drug delivery with controlled release and in particular delivery of gene therapy drugs.

### SUMMARY OF THE INVENTION

Disclosed herein are novel targeted drug delivery and controlled release methods and compositions where optically absorbing nanoparticles, such as nanoshells, are functionalized on their surfaces with thermolabile molecules that bind the drug molecules to be delivered. The linkage between the thermolabile molecule on the nanoshell and the drug is deliberately designed or selected to be thermal labile, so that upon illumination of the nanoparticle at a wavelength of light, the drug molecules on the nanoparticles will be released. Targeting molecules, such as antibodies, aptamers or other molecules like folic acid, can be concurrently bound to the nanoparticle surface to deliver the nanoparticle to specifically targeted cells or tissues prior to the photothermally induced drug release. In this way the nanoparticles can be advantageously concentrated on the target prior to illumination, which makes the disclosed compositions both a targeted delivery and a controllable drug release vehicle. Further features and advantages of the disclosed compositions and methods will be described in more detail below.

In an embodiment, a composition for delivering one or more drugs comprises one or more nanoparticles. The one or more nanoparticles convert electromagnetic radiation into heat energy when the nanoparticles are irradiated with electromagnetic radiation. The composition also comprises one or more thermolabile moieties coupled to each nanoparticle. In addition, the composition comprises one or more drug molecules coupled to the one or more thermolabile moieties through thermolabile interactions. The one or more thermolabile moieties are temperature sensitive so as to release the one or more drugs upon exposure to heat from the one or more nanoparticles.

In another embodiment, a composition for delivering one or more drugs comprises one or more nanoparticles. The one or more nanoparticles convert electromagnetic radiation into heat energy when the nanoparticles are irradiated with electromagnetic radiation. The composition additionally comprises one or more thermolabile moieties coupled to each nanoparticle. Furthermore, the composition comprises one or more drug molecules coupled to the one or more thermolabile moieties through thermolabile interactions. The one or more thermolabile moieties are temperature sensitive so as to release the one or more drugs upon exposure to heat from said one or more nanoparticles. The composition also comprises one or more targeting molecules covalently attached to the one or more nanoparticles, either via the thermolabile moieties or directly to the nanoparticle surface.

In another embodiment, a method comprises providing a plurality of nanoparticles having at least one thermolabile moiety coupled to each nanoparticle. The nanoparticles convert incident radiation into heat energy when said nanoparticles are irradiated with electromagnetic radiation. The method also comprises binding the one or more drugs to the at least one thermolabile moiety through thermolabile interactions.

The foregoing has outlined rather broadly the features and technical advantages of the present invention in order that the detailed description of the invention that follows may be better understood. Additional features and advantages of the invention will be described hereinafter that form the subject of the claims of the invention. It should be appreciated by those skilled in the art that the conception and the specific embodiments disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims.

### BRIEF DESCRIPTION OF THE DRAWINGS

For a detailed description of the embodiments of the invention, reference will now be made to the accompanying drawings in which:

FIG. 1 illustrates an embodiment of a composition and a method for delivering one or more drugs;

FIG. 2 illustrates an embodiment of a composition for delivering one or more drugs. K represents a lysine repeating unit;

FIG. 3 shows fluorescent micrographs of cells incubated with the nanoshell-polylysine conjugate bound to a green fluorescent protein short interfering RNA (GFP siRNA) as compared to controls without GFP siRNA and scrambled siRNA;

FIG. 4 is chart of relative green fluorescent protein expression for (i) nanoshell-polylysine conjugate (NS-PLL), and (ii) NS-PLL conjugate with antibody (EGFR Ab) irradiated with two different illumination times and also without laser irradiation; and

FIG. 5 is a chart of the measured ratio of green fluorescent protein intensity to 4′,6-diamidino-2-phenylindole intensity for cells incubated with (i) NS-PLL-short interfering RNA (siRNA) precipitate, (ii) NS-PLL-siRNA supernatant, (iii) siRNA by itself, and (iv) blank control.

### NOTATION AND NOMENCLATURE

Certain terms are used throughout the following description and claims to refer to particular system components. This document does not intend to distinguish between components that differ in name but not function.

In the following discussion and in the claims, the terms “including” and “comprising” are used in an open-ended fashion, and thus should be interpreted to mean “including, but not limited to . . . ”. The term “coupled” means a direct or indirect connection or bond.

### DETAILED DESCRIPTION OF THE PREFERRED EMBODIMENTS

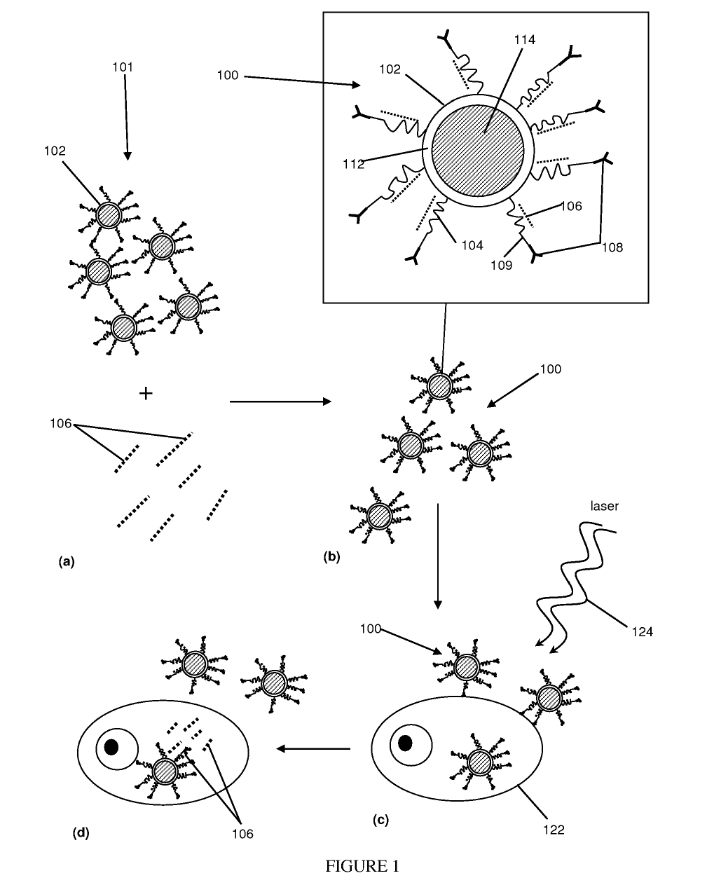

FIG. 1 illustrates an embodiment of a method and a composition 100 for delivering one or more drugs. In general, the composition 100 includes one or more nanoparticles 102 having one or more thermolabile moieties 104 attached to the surface of the one or more nanoparticles. In addition, the composition 100 comprises one or more drug molecules 106 attached to nanoparticles 102 via the thermolabile moieties 104 through thermolabile interactions such that a change in temperature triggers releases of the one or more drugs 106 from the nanoparticles 102.

As used herein, a “thermolabile moiety” refers to any suitable molecular entity that: (a) covalently binds to the nanoparticle 102 surface, and (b) provides a thermolabile interaction with the drug or drugs to be delivered (e.g., the binding between the thermolabile moiety 104 and the drug 106 is no longer sufficient to hold drug at elevated temperature). Furthermore, as used herein, “thermolabile” refers to any compound which is subject to change in response to heat. In addition, the term “thermolabile interaction(s)” refers to any non-covalent binding to a compound which is temperature sensitive including without limitation, chemical/physical effects such as hydrophobic, steric (dangling), electrostatic, hydrogen bonding, van de Waals forces, dipole-dipole bonding, ionic bonds, or any combination of these. In other words, thermolabile moiety 104 binds a therapeutic compound or drug at one temperature, but then releases the drug at a different temperature. The composition 100 may also comprise one or more targeting moieties or molecules 108 coupled to the nanoparticles 102.

The nanoparticles 102 preferably comprise nanoparticles 102 known to those of skill in the art that are capable of converting radiation into heat when exposed to electromagnetic radiation such as light. The electromagnetic radiation may be encompass any wavelength of radiation including without limitation, x-rays, UV light, IR light, visible light, or combinations thereof.

In an embodiment, the one or more nanoparticles 102 comprise nanoshells. The nanoshells may comprise a dielectric core 114 and outer conductive shell 112. The diameter of a nanoshell 12 can exceed the nanometer range, extending from about 1 nm to about 5 microns. As more fully described in U.S. Pat. No. 6,344,272, which is herein incorporated by reference in its entirety for all purposes, metal nanoshells manifest unique physical properties. Specifically, metal nanoshells possess optical properties similar to metal colloids, i.e., a strong optical absorption and an extremely large and fast third-order nonlinear optical (NLO) polarizability associated with their plasmon resonance.

At resonance, dilute solutions of gold colloid possess some of the strongest electronic NLO susceptibilities of any known substance. Metal nanoshells exhibit similarly strong electronic NLO susceptibilities. However, unlike simple metal colloids which each have only a single resonance frequencies, the plasmon resonance frequency of metal nanoshells depends on the relative size of the nanoparticle core and the thickness of the metallic shell. By adjusting the relative core and shell thickness, metal nanoshells can be fabricated that will absorb or scatter light at any wavelength across the entire visible and infrared range of the electromagnetic spectrum. The relative size or depth of the particle's constituent layers determines the wavelength of its absorption. Whether the particle absorbs or scatters incident radiation depends on the ratio of the particle diameter to the wavelength of the incident light.

For any given core 114 and shell 112 materials, the maximum absorbance of the particle depends upon the ratio of the thickness (i.e., radius) of the core to the thickness of the shell. Based on the core radius:shell thickness (core:shell) ratios that are achieved by the referenced synthesis method, nanoshells manifesting plasmon resonances extending from the visible region to approximately 5 μm in the infrared can be readily fabricated. By varying the conditions of the metal deposition reaction, the ratio of the thickness of the metal shell to the core radius can be varied in a predictable and controlled way. Accordingly, particles are constructed with core radius to shell thick ratios ranging from about 2-1000. This large ratio range coupled with control over the core size results in a particle that has a large, frequency-agile absorbance over most of the visible and infrared regions of the spectrum.

By comparison, the shifts induced in the plasmon resonance of gold colloid by adsorption of molecular species are quite small, typically 10 nm or less. The nonlinear optical (NLO) properties of metal nanoshells or nanoshells-constituent materials can be resonantly enhanced by judicious placement of the plasmon resonance at or near the optical wavelengths of interest. The extremely agile “tunability” of the plasmon resonance is a property completely unique to metal nanoshells. In no other molecular or nanoparticle structure can the resonance of the optical absorption and NLO properties be systematically designed, much less so easily and over such an extremely wide range of wavelengths.

As described in U.S. Pat. No. 6,344,272, a more generalized method for the growth of a uniform metallic layer of nanometer scale thickness onto a dielectric core has been developed. Briefly described, the process includes growing or obtaining dielectric or semiconductor nanoparticles dispersed in solution. Very small (i.e., 1-2 nm) metal “seed” colloid is tethered or attached to the surface of the nanoparticles 102, preferably via chemical or molecular linkages joining the shell layer to the dielectric core layer. Suitable linker molecules include any molecule that is capable of binding both the core and atoms, ions or molecules of the shell. Preferably, linker binding is covalent to both the shell and the inner layer, but binding may also be through ionic bonds, lone-pair interactions, hydrogen bonds, Van der Waals interaction or the like. The seed colloids cover the dielectric nanoparticle surfaces with a discontinuous metal colloid layer. Additional metal is then grown onto the “seed” metal colloid adsorbates by chemical reduction in solution. This approach has been successfully used to grow both gold and silver metallic shells onto silica nanoparticles.

Suitable metals for forming the shell or outer layer 112 include the noble and coinage metals, but other electrically conductive metals may also be employed, the particular choice depending upon the desired use. Metals that are particularly well suited for use in shells include, but are not limited to, gold, silver, copper, platinum, palladium, lead, iron and the like. Gold and silver are preferred. Alloys or non-homogenous mixtures of such metals may also be used. The shell layer is preferably about 1 to 100 nm thick and coats the outer surface of the core uniformly, or it may partially coat the core with atomic or molecular clusters.

The core 114 may have a spherical, cubical, cylindrical or other shape. Regardless of the geometry of the core 114, it is preferred that the particles be substantially homogeneous in size and shape, and preferably spherical. Preferably compositions comprising a plurality of metal nanoshells will contain particles of substantially uniform diameter ranging up to several microns, depending upon the desired absorbance maximum of the particles.

Preferably, the core 114 or adjacent inner layer to the shell layer be nonconducting or dielectric. Suitable dielectric core materials include, but are not limited to, silicon dioxide, iron oxide, gold sulfide, titanium dioxide, polymethyl methacrylate (PMMA), polystyrene, and macromolecules such as dendrimers. The material of the nonconducting layer influences the properties of the particle, so the dielectric constant of the core material affects the absorbance characteristics of the particle. The core 114 may be a mixed or layered combination of dielectric materials.

The particles 100 employed may be two-layered, having a nonconducting core 114 and a conducting outer layer or shell 112. If desired, an optically tuned multiwalled or multilayer nanoshell particle may be formed by alternating nonconducting and conducting layers. It is preferred that at least one shell layer readily conduct electricity, however, in some cases it is only necessary that one shell layer have a lower dielectric constant than the adjacent inner layer. The metal or metal-like shell layer is preferably the outermost layer. Additional layers may be attached to the adjacent layers using the methods described herein. The particles preferably have a homogeneous radius that can range from approximately 1 nanometers to several microns, depending upon the desired absorbance maximum of the embodiment.

An example of one embodiment of suitable nanoshells is as follows. Gold nanoshells with a 37 nm average diameter gold sulfide core and a gold shell average thickness of 4 nm were formed by combining 20 ml of 2 mM HAuCl₄ and 28 ml of 1 mM Na₂S. The progress of the reaction was monitored using an UV-visible spectrophotometer (U-2001, Hitachi Co., Tokyo) to observe the extinction spectrum of the solution from 400-1050 nm. As the nanoshells formed, the extinction spectra exhibited a peak that red-shifted into the IR, then halted and began to blue-shift into the visible spectrum. The peak narrowed and increased in magnitude as this occurred. 3.5.quadrature.1 of mercaptoproprionic acid was added to halt this shift, by halting the growth of the gold shell, when the extinction peak reached the desired wavelength. For example, in one preparation where the wavelength of the laser to be used with the nanoshells was about 1064 nm, the growth of the gold shell was arrested when the extinction peak was centered around 1050 nm. The solution was then brought to pH 10.5 with 1 M NaOH, centrifuged at 3000 RPM for 20 minutes four times, and stored at 4° C. The size and polydispersity of the resulting nanoshells were determined by evaporating a drop of the nanoshell solution onto a carbon film on a copper grid and viewing the nanoshells via transmission electron microscopy (TEM, JEM-2010, JEOL, Peabody, Mass.). Other nanoshell preparations having a maximum absorbance wavelength of about 821 nm were prepared similarly. The maximum absorbance wavelength of the nanoshells is preferably within about 10-15 nm of the peak wavelength of the excitation laser to be employed. The gold/gold sulfide nanoshells made in this way could be formed with desired coating thicknesses and thus were tunable over a range of about 600-1,100 nm.

In other embodiments, the nanoparticles 102 comprise nanoshells, nanorice, nanoeggs, nanorods, nanospheres, or combinations thereof. For example, the nanoparticles 102 may comprise solid metal nanoparticles. Thus, any suitable nanoparticle known to those of skill in the art may be used in conjunction with the disclosed composition.

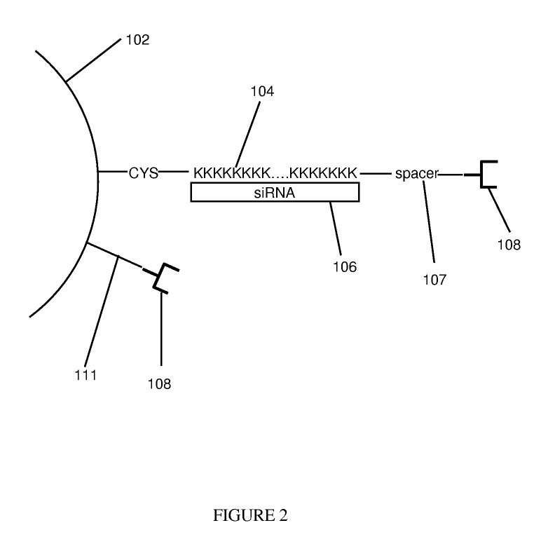

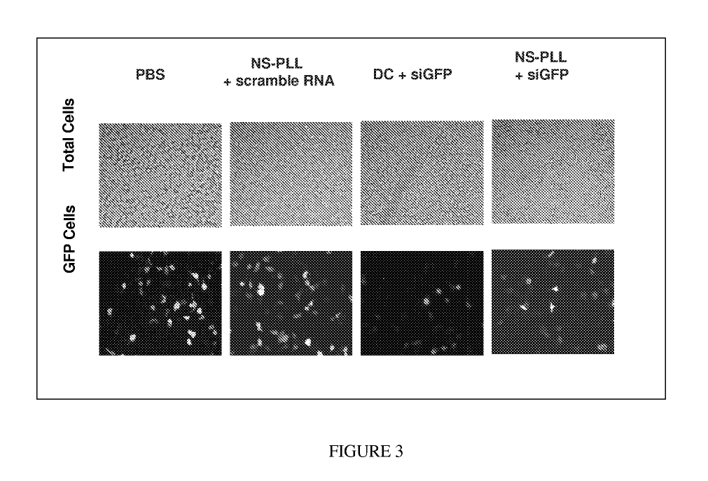

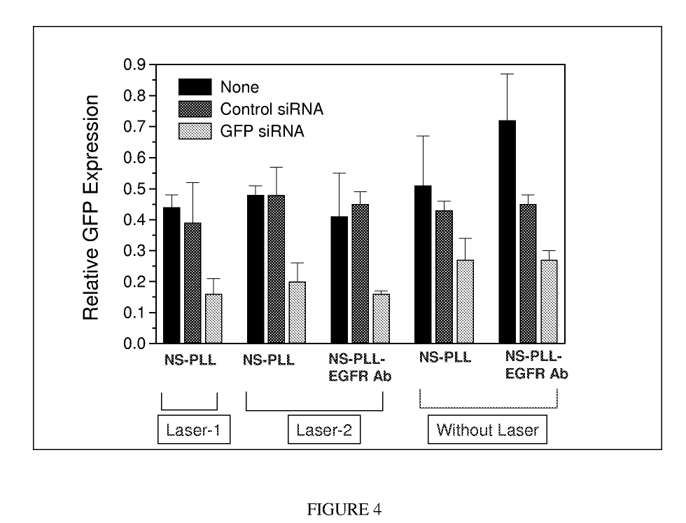

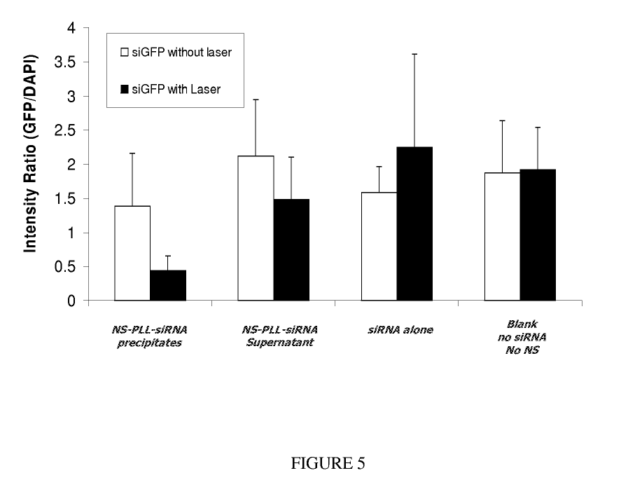

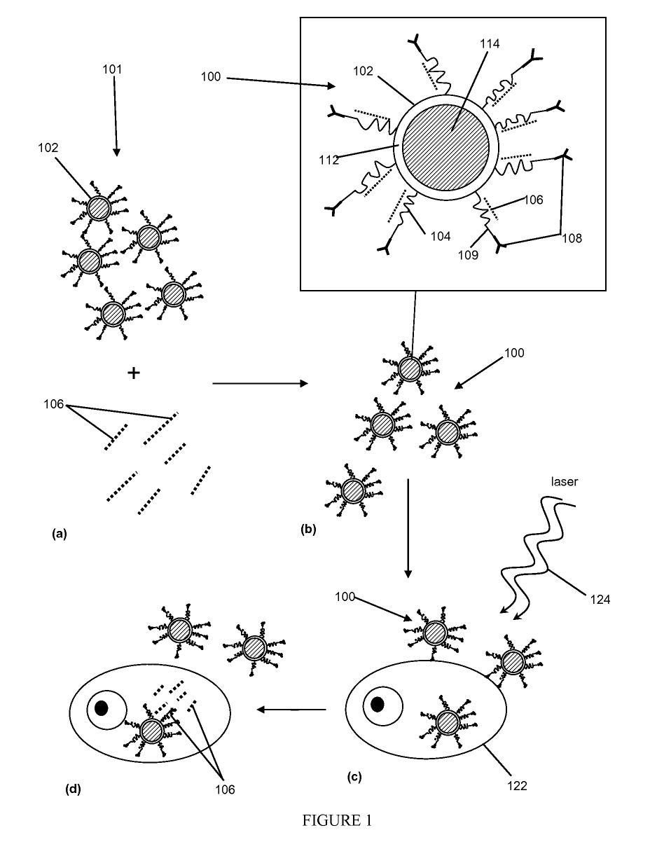

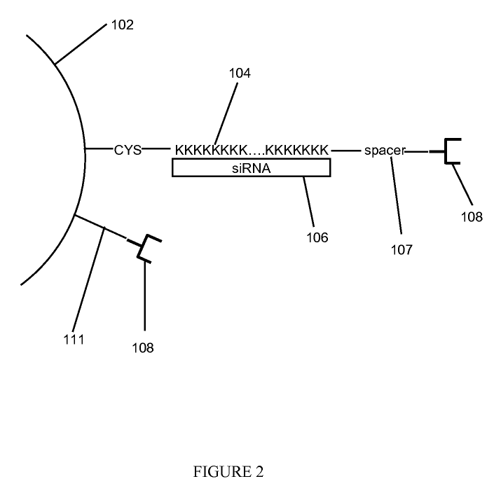

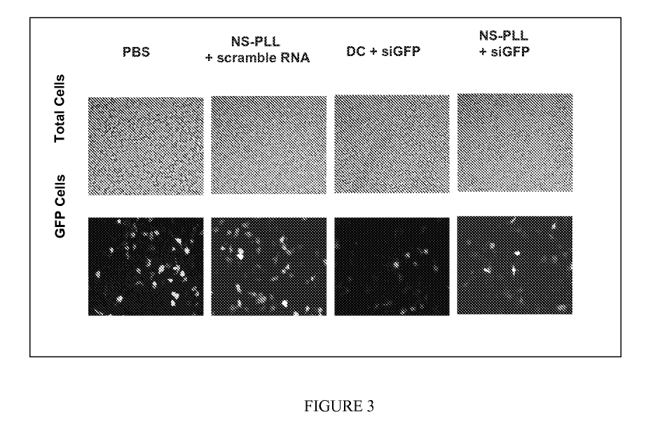

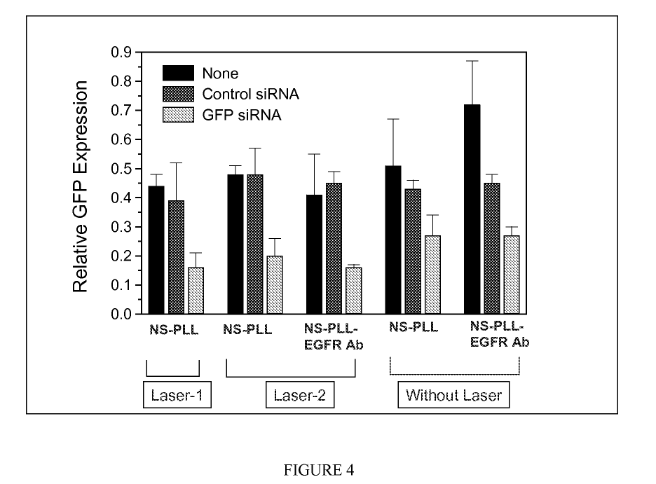

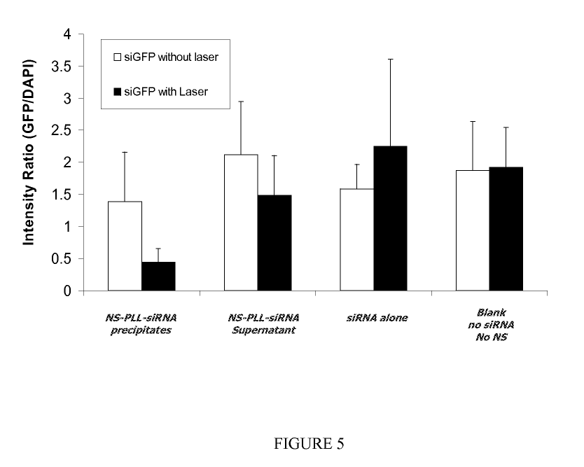

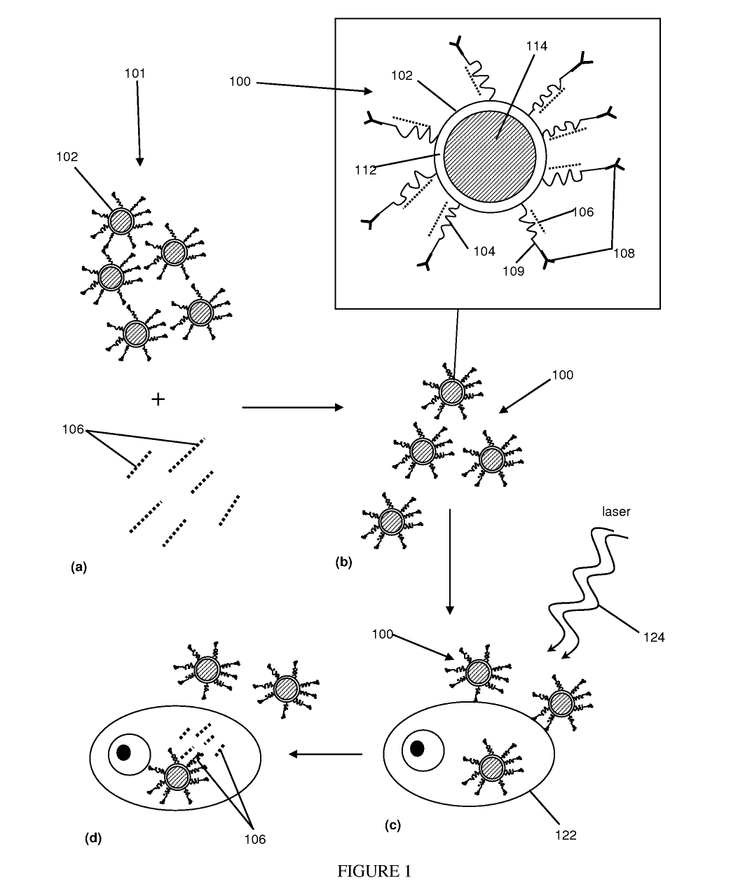

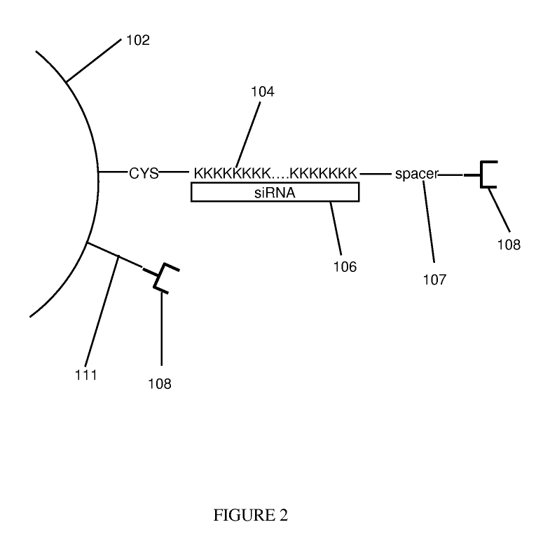

Referring now to FIG. 2, in an embodiment, one or more thermolabile moieties 104 are coupled to the surface of the nanoparticles 102. The thermolabile moieties 104 are generally covalently attached to nanoparticles 102. By way of example only, thermolabile moieties 104 may be covalently bound to the nanoparticle surface using a sulfide-gold linkage by mixing cysteine-containing peptides with the nanoparticles 102. Another example of a covalent linkage may be a cysteine-tyrosine-serine (CYS) linker as shown in FIG. 2. However, thermolabile moieties 104 may be bound to the nanoparticle surface using any techniques or compounds known to those of skill in the art. Examples of thermolabile moieties include without limitation, polycations, graft copolymers, single stranded DNA, double stranded DNA, RNA, or combinations thereof. Furthermore, examples of suitable polycations that may be used as the thermolabile moiety include without limitation, polylysine, polyethyleneimine, poly(amido amine)s, chitosan, poly(amino ester)s, any copolymers that contain amine groups (primary, secondary and tertiary), or combinations thereof. In an embodiment, the thermolabile moiety 104 is poly L-lysine (PLL). PLL of any molecular weight may be used as the thermal labile moiety 104. More particularly, the PLL may have molecular weights ranging from about 500 Da to about 100,000 Da, alternatively ranging from about 2,000 to about 50,000 Da, alternatively from about 1,000 Da to about 10,000 Da.

Furthermore, different types or molecular weights of thermolabile moieties 104 may be bound to the nanoparticles 102 to provide moieties 104 with different temperature sensitivities. For example, two different molecular weights of PLL may be attached to the nanoparticles 102 such that composition 100 may release drugs 106 at two different temperatures. Alternatively, a different polycation other than PLL may be used to provide the difference in temperature sensitivity.

The thermolabile moieties 104 may be conjugated with various other molecules. For example, poly(ethylene glycol) may be conjugated or coupled to the thermolabile moieties 104 to act as a spacer 109 either between the moiety 104 and the nanoparticle 102 or between the moiety 104 and the targeting moiety 108. Other examples of suitable conjugates with the thermolabile moieties 104 include without limitation, lipids, proteins, antibodies, peptides, cholesterol, or combinations thereof.

Although the disclosed compositions are generally applicable to targeted drug delivery and release for any drug 106, it may also be particularly advantageous for gene, antisense, or aptamer (DNA and RNA) therapy. Thus, in some embodiments, the thermolabile molecules 104 may be advantageously designed to be complementary to DNA or RNA oligomers as drugs 106, so that DNA strands will be delivered to the target site in a far more inert form of double stranded DNA or a DNA-RNA complex. Without being limited by theory, it is believed that this will greatly prolong the circulation time of the delivered DNA or RNA, allowing far greater drug concentrations to be delivered to the target site. In another embodiment, the one or more drugs 106 are short interfering RNAs (siRNA) which are coupled to the thermolabile moieties by thermolabile interactions. Small interfering RNA, sometimes known as short interfering RNA or silencing RNA, are a class of 20-25 nucleotide-long double-stranded RNA molecules that play a variety of roles in biology. Most notably, siRNA may be involved in the RNA interference (RNAi) pathway where the siRNA interferes with the expression of a specific gene. In addition to their role in the RNAi pathway, siRNAs also act in RNAi-related pathways, e.g., as an antiviral mechanism or in shaping the chromatin structure of a genome.

In other embodiments, any type of drug 106 may be bound by the thermolabile molecules 104. These include various therapeutic compounds such as for chemotherapy. Other examples include without limitation, a chemotherapeutic drug, insulin, an antibiotic, or combinations thereof.

As noted above, the drugs 106 are preferably bound to the thermolabile moieties 104 by thermolabile interactions. It is emphasized that the bond between the thermolabile moieties 104 and drugs (e.g., siRNA or ss DNA) is not a covalent bond, but a thermolabile bond such as without limitation, chemical/physical effects such as hydrophobic, steric (dangling), electrostatic, hydrogen bonding, van de Waals forces, dipole-dipole bonding, ionic bonds, or any combination of these.

In a further embodiment, one or more targeting molecules 108 or moieties are coupled to the nanoparticles 102. Targeting molecules 108 may be attached to the nanoparticles 102 via thermolabile moiety 104 as shown in FIG. 2. A spacer 109 may be disposed between targeting molecule 108 and thermolabile moiety 104. Without being limited by theory, spacer 109 may provide thermal protection to the attached drug 106 and also to the targeted tissue or cell 122. In an embodiment, spacer 109 may be a polymer. Thus, spacer length may be adjusted by using polymers having different chain lengths and have different properties using different monomers. Other examples of suitable spacers 109 include without limitation, proteins, polypeptides, oligopeptides, or combinations thereof. Targeting moieties 108 may also covalently attached directly to the surface of nanoparticles 102. As shown in FIG. 2, targeting moieties 108 may be directly attached to surface of nanoparticle 102 through a linking molecule 111. Alternatively, targeting moieties 108 themselves may be attached to surface of nanoparticle 102.

The targeting moieties 108 may be any compounds or chemical which show preferential binding to specific proteins, receptors, and/or chemicals. In embodiments where more than one targeting molecule 108 is attached to each nanoparticle, each targeting moiety 108 may be the same or different. Targeting moieties 108 are preferably tailored to a desired target tissue. For example, the molecules 108 may be specific for nerve tissue, heart tissue, vascular tissue, lung tissue, or any other organ of the body. Examples of suitable targeting moieties 108 include without limitation, an antibody, a peptide sequence, a protein, an oligopeptide sequence, a DNA oligomer, a RNA oligomer, small molecules such as folic acid or combinations thereof.

FIG. 1 shows a schematic of an embodiment of a method for delivering one or more drugs. In this embodiment, the molecular carrier(s) 104 and the targeting moiety 108 or recognition reagent are covalently attached to the nanoparticle to form conjugated nanoparticles 101 as seen in (a). As noted above, suitable thermolabile moieties 104 include without limitation, DNA, RNA oligomers, polymers, or any combination of chemicals which can be released by thermal stimulation of the labile interaction between the drug 106 and thermolabile molecule 104. These molecules may be attached to the nanoparticles 102 by any method known to those of skill in the art. As discussed above, the targeting moiety 104 can be aptamer, antibody or combinations thereof.

Once the functional moieties or molecules are attached or coupled to the nanoparticles 102, the one or more drugs 106 are bound to the thermolabile moieties 104 in (b) to form the drug delivery composition 100. Typically, one type of drug is bound to the nanoparticles 102 via the thermolabile moieties 104. However, in one embodiment, each thermolabile moiety 104 may be bound to the same drug or a different type of drug 106 allowing the delivery of a combination of drugs. For example, two different types of siRNA may be bound to the same or different thermolabile moieties 104 such that each type of siRNA may provide a different type of gene therapy.

The nanoparticles 102, once loaded with drugs, may be delivered to the tissue site in (c) where the targeting moieties 108 bind to the target cells 122. In an embodiment, the disclosed compositions 100 may be injected into the bloodstream for targeted drug delivery or may be placed in close proximity in situ, such as without limitation, injection or surgical procedure. After delivering the functionalized nanoparticle 101 to the tissue or cells 122, the composition 100 accumulate on the targeted tissues or cells 122 as shown in FIG. 1(c). Alternatively, composition 100 may be up taken or phagocytosed by cells 122 as also shown in FIG. 1(c). By illuminating the area where the drug is targeted with electromagnetic radiation (e.g., light) 124, the drug 106 on the nanoshell 102 is released because of the laser thermal effect as shown in (d). In a specific embodiment, the composition 100 may be irradiated with laser light having a wavelength ranging from about 100 nm to about 5,000 nm, alternatively from about 200 nm to about 3,000 nm, alternatively from about 800 nm to about 1,100 nm. During the irradiation, the ambient temperature of the tissue remains at a temperature of less than 80° C., alternatively less than about 60° C., alternatively less than about 50° C. The laser radiation 123 may be pulsed or continuous. Additionally, the composition 100 may be irradiated for any suitable time period by the laser. Once released, the drugs 106 may be up taken by the tissue or cells 122 or released within cells 122 where the therapeutic effect of the drugs 106 may take effect as shown in (d).

This nanoparticle based delivery method is suitable to a very wide range of medical and biomedical applications, including gene and antisense therapy, multiple modality contrast enhancement in bioimaging, etc. The disclosed compositions and methods may greatly enhance therapeutic efficacy of chemical and biochemical pharmaceuticals, and/or reduce the cytotoxicity of the delivered drugs by the targeted and controlled release of the thermolabile moieties.

### EXAMPLE

Thermolabile nanoparticles for gene delivery were synthesized using the following procedure. 2 μM of poly L-lysine was conjugated with about 10 μM of gold nanoshells. The gold nanoshells were fabricated in accordance with methods and techniques described elsewhere (see e.g., U.S. Pat. No. 6,344,272). The mixture was centrifuged and diluted for three cycles. After centrifugation and dilution about 100 nM of siRNA was added to the conjugated nanoshells. The siRNA was specific to the GFP gene. That is, the siRNA theoretically should bind to the GFP gene and prevent production of the GFP protein. The siRNA and nanoshells were allowed to incubate overnight.

The siRNA-nanoshell mixture was centrifuged and the supernatant removed to remove any siRNA which did not bind to the nanoshell conjugates. The siRNA-PLL-nanoshell precipitate was then mixed with culture medium and green fluorescent protein (GFP) transfected H1299 (a lung cancer cell line) cells, and incubated overnight.

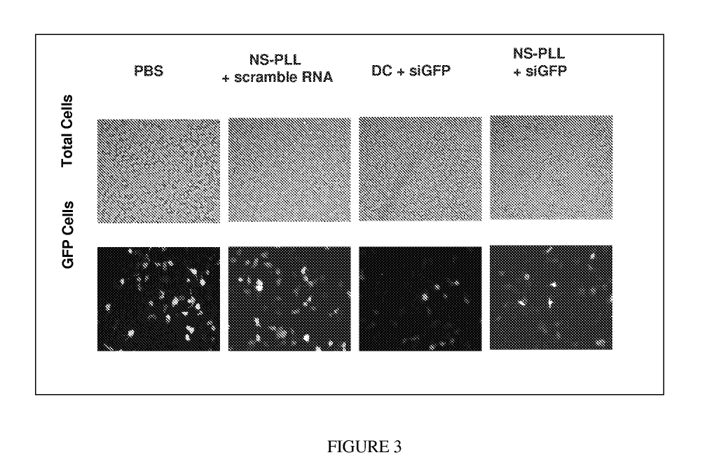

Fluorescence intensity of the cell nucleus was measured using 4′,6-diamidino-2-phenylindole stain (DAPI) while fluorescence intensity of the GFP was also determined. The ratio of GFP to DAPI was then calculated for the following samples: siRNA-PLL-nanoshell precipitate, siRNA-PLL-nanoshell supernatant, samples with siRNA alone, and control samples without nanoshells or siRNA. Each sample was measured with and without laser irradiation. A Ti-sapphire femto-second pulse laser with excitation wavelength around 800 nm was used to irradiate the cells. Results are shown in FIGS. 3-5. A lower ratio indicated better effectiveness of siRNA delivery.

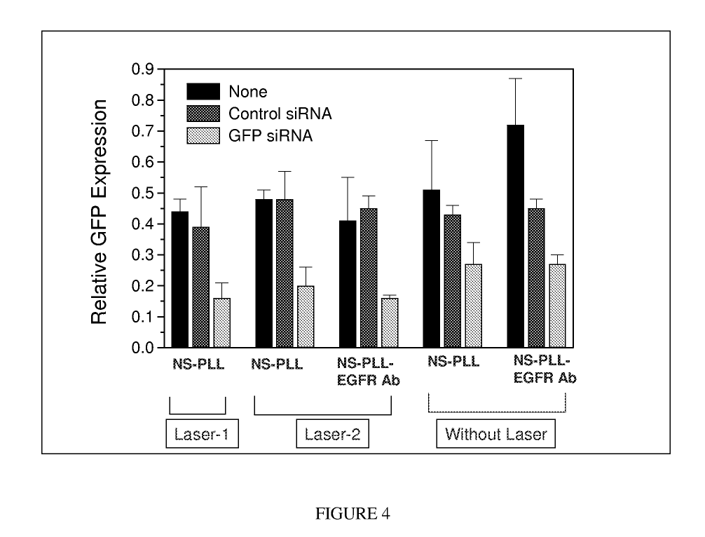

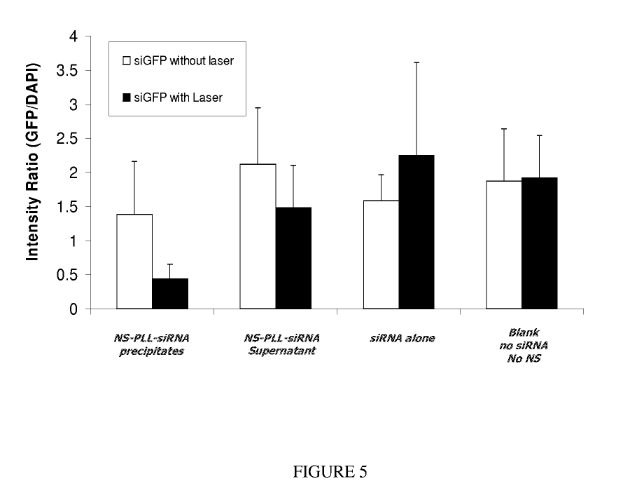

FIG. 3 shows fluorescent images of cells incubated with phosphate buffered saline (PBS) controls, cells incubated with siRNA-PLL-nanoshells having a scrambled RNA attached, cells incubated with polymer micelle-anti-GFP siRNA, and cells incubated with siRNA-PLL-nanoshells having anti-GFP RNA attached. As seen in FIG. 3, cells incubated with siRNA-PLL-nanoshells having anti-GFP RNA show significantly decreased amount of green fluorescence when compared to the control samples. As can be seen in FIGS. 4 and 5, samples containing siRNA-PLL-nanoshell precipitate and irradiated with a laser showed significantly lower expression of GFP than the other control samples or samples that were not irradiated with laser radiation. In FIG. 5, the siRNA-PLL-nanoshell supernatant did not show any de-activation of the GFP gene indicating that most of the siRNA was bound to the siRNA-PLL-nanoshell composition. Accordingly, these experiments showed that the siRNA was released from the thermolabile moieties (PLL) by heating of the nanoshells by the laser and further, that the released siRNA molecules were able to de-activate the GFP gene.

While embodiments of the invention have been shown and described, modifications thereof can be made by one skilled in the art without departing from the spirit and teachings of the invention. The embodiments described and the examples provided herein are exemplary only, and are not intended to be limiting. Many variations and modifications of the invention disclosed herein are possible and are within the scope of the invention. Accordingly, the scope of protection is not limited by the description set out above, but is only limited by the claims which follow, that scope including all equivalents of the subject matter of the claims.

The discussion of a reference in the Description of the Related Art is not an admission that it is prior art to the present invention, especially any reference that may have a publication date after the priority date of this application. The disclosures of all patents, patent applications, and publications cited herein are hereby incorporated herein by reference in their entirety, to the extent that they provide exemplary, procedural, or other details supplementary to those set forth herein.

1. A composition for delivering one or more drugs comprising:
one or more nanoparticles, wherein said one or more nanoparticles convert electromagnetic radiation into heat energy when said nanoparticles are irradiated with electromagnetic radiation;
one or more thermolabile moieties coupled to each nanoparticle; and
one or more drug molecules coupled to said one or more thermolabile moieties through thermolabile interactions, wherein said one or more thermolabile moieties release the one or more drugs upon exposure to heat from said one or more nanoparticles.

2. The composition of claim 1 wherein said one or more nanoparticles comprise nanoshells, nanorice, nanoeggs, nanorods, nanospheres, or combinations thereof.

3. The composition of claim 1 wherein each nanoparticle comprises a dielectric core surrounded by a conductive shell.

4. The composition of claim 1 wherein said one or more nanoparticles are gold nanoshells.

5. The composition of claim 1 wherein said one or more thermolabile moieties comprises a polycation, a graft copolymer, single stranded DNA, double stranded DNA, RNA, or combinations thereof.

6. The composition of claim 5 wherein said polycation comprises polylysine, polyethyleneimine, poly(amido amine)s, chitosan, poly(amino ester)s, copolymers containing amine groups, or combinations thereof.

7. The composition of claim 1 wherein said one or more drug molecules comprise at least two different drug molecules.

8. The composition of claim 1 further comprising one or more targeting moieties coupled to each nanoparticle.

9. The composition of claim 8 wherein said one or more targeting moieties comprises an antibody, a peptide sequence, a protein, an oligopeptide sequence, a DNA oligomer, a RNA oligomer, folic acid, or combinations thereof.

10. The composition of claim 8 wherein said one or more targeting moieties are directly coupled to the surface of each nanoparticle.

11. The composition of claim 8 wherein said one or more targeting moieties are coupled to each nanoparticle by said one or more thermolabile moieties.

12. The composition of claim 11 further comprising a spacer disposed between said one or more targeting moieties and said one or more thermolabile moieties.

13. The composition of claim 12 wherein said spacer comprises a polymer, a protein, a polypeptide, or combinations thereof.

14. The composition of claim 1 wherein said one or more drug molecules comprise DNA, RNA, a polymer, a chemotherapeutic drug, insulin, an antibiotic, a short interfering RNA (siRNA), an anti-sense DNA, or combinations thereof.

15. The composition of claim 1 wherein said one or more drug molecules comprise two different siRNA molecules.

16. The composition of claim 1 wherein said thermolabile interactions comprise electrostatic interactions, hydrogen bonds, Van der Waals forces, ionic bonds, dipole-dipole bonds, or combinations thereof.

17. Use of the composition of claim 1 for gene therapy.

18. Use of the composition of claim 1 for treatment of genetic diseases.

19. Use of the composition of claim 1 for bioimaging.

20. A composition for delivering one or more drugs comprising:
one or more nanoparticles, wherein said one or more nanoparticles convert electromagnetic radiation into heat energy when said nanoparticles are irradiated with electromagnetic radiation;
one or more thermolabile moieties coupled to each nanoparticle;
one or more drug molecules coupled to said one or more thermolabile moieties through thermolabile interactions, wherein said one or more thermolabile moieties are temperature sensitive so as to release the one or more drugs upon exposure to heat from said one or more nanoparticles; and
one or more targeting molecules covalently attached to said one or more nanoparticles.

21. A method comprising:
a) providing a plurality of nanoparticles having at least one thermolabile moiety coupled to each nanoparticle, wherein the nanoparticles convert incident radiation into heat energy when said nanoparticles are irradiated with electromagnetic radiation; and
b) binding the one or more drugs to the at least one thermolabile moiety through thermolabile interactions.

22. The method of claim 21, further comprising:
c) delivering the plurality of nanoparticles to a tissue; and
d) exposing the plurality of nanoparticles to electromagnetic radiation to release the one or more drug molecules from the nanoparticles and deliver the one or more drugs to the tissue.

23. The method of claim 22 wherein (c) comprise injecting the nanoparticles into the tissue.

24. The method of claim 22 wherein (d) comprises exposing the plurality of nanoparticles to light having a wavelength ranging from about 800 nm to about 1,100 nm.

25. The method of claim 22 wherein exposing the plurality of nanoparticles to electromagnetic radiation in (d) causes the temperature of the tissue increase to a temperature of no more than about 50° C.

26. The method of claim 21 wherein each nanoparticle is further coupled to one or more targeting moieties.

27. The method of claim 26 wherein the one or more targeting molecules exclusively bind to the tissue.

28. The method of claim 21 wherein the plurality of nanoparticles are nanoshells.

29. The method of claim 21 wherein the one or more thermolabile moieties comprise polycations, graft copolymers, single stranded DNA, double stranded DNA, RNA, or combinations thereof.
